(19) United States
(12) Patent Application Publication
(10) Pub. No.: US 2006/0206183 A1
(43) Pub. Date: Sep. 14, 2006

(54) SPINAL CORD STIMULATOR LEAD FOR NEUROSTIMULATION HAVING A FLUID DELIVERY LUMEN AND/0R A DISTENSIBLE BALLOON

(76) Inventors: Stephen T. PYLES, Ocala, FL (US); Daniel A. Graubert, Etna, NH (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/421,098

(22) Filed: May 31, 2006

Related U.S. Application Data
(63) Continuation-in-part of application No. 11/217,061, filed on Aug. 31, 2005.
(60) Provisional application No. 60/606,172, filed on Aug. 31, 2004.

(51) Int. Cl. A61N 1/05

(52) U.S. Cl. 607/117; 607/43

(57) ABSTRACT

A spinel cord stimulator lead for placement in the epidural space of a human or animal subject. The spinal cord stimulator lead includes a biocompatible body portion defining an elongate shaft, wherein at least a portion of which is flexible; at least one electrode positioned along the shaft; a lumen extending through at least a portion of the shaft for carrying a fluid; and a distensible balloon positioned around a distal end of the shaft and in fluid communication with the lumen. Preferably, the balloon is a cuffed balloon that expands radially outwardly from at least a portion of the shaft's distal end. The spinal cord stimulator lead can also include a second lumen for discharging a fluid directly to a tissue obstruction and a stylet for guiding the stimulator lead into and through the epidural space. The spinal cord stimulator lead can have the form of a percutaneous lead or a surgical lead.

### CROSS-REFERENCE TO RELATED APPLICATIONS

This application is continuation-in-part of U.S. Non-Provisional patent application Ser. No. 11/217,061, filed Aug. 31, 2005, which claims priority to U.S. Provisional Patent Application Ser. No. 60/606,172, filed Aug. 31, 2004, which are hereby incorporated herein by reference in their entireties for all purposes.

### TECHNICAL FIELD

The present invention relates generally to the field of surgical instruments and methods, and more particularly to a spinal cord stimulator lead having a fluid delivery lumen and a plurality of electrodes for neurostimulation.

### BACKGROUND OF THE INVENTION

Spinal cord stimulation is used to alleviate chronic pain by stimulating the central nervous system. Conventional spinal cord stimulator leads include percutaneous leads and surgical leads. Percutaneous leads, such as the Medtronic PISCES-Quad® or Octad® leads or the ANS Octrode® and Quattrode® leads, are solid and have a plurality, but typically four or eight, electrodes. The percutaneous leads can be inserted through a needle and placed in the epidural space, in close proximity to the spinal cord. When activated, the electrodes deliver a precise, mild electrical impulse to the spinal cord or to a peripheral nerve. The electrical impulses activate pain inhibitory mechanisms to block the pain signal from reaching the brain.

However, accurately placing known electrodes can be rather difficult because the epidural space that surrounds the spinal cord typically contains fat, veins, adhesions, and connective tissue membranes which interfere with, and often prevent, the accurate placement of the electrodes.

Therefore, a need exists for an apparatus and method which would allow for greater ease in placing percutaneous electrodes in the epidural space.

### SUMMARY OF THE INVENTION

In an example form, the present invention is a spinal cord stimulator lead for placement in the epidural space of a human or animal subject. The stimulator lead includes a biocompatible body portion defining an elongate shaft, wherein at least a portion of which is flexible; at least one electrode positioned along the shaft; a lumen extending through at least a portion of the shaft for carrying a fluid; and a distensible balloon positioned around a distal end of the shaft and in fluid communication with the lumen. Preferably, the balloon is a cuffed balloon that expands radially outwardly from at least a portion of the shaft's distal end. Also preferably, the lumen carries a sterilized fluid under sufficient pressure to expand the balloon. Additionally, the spinal cord stimulator lead can include a stylet for guiding the stimulator lead into and through the epidural space. The spinal cord stimulator lead can further include a second lumen for discharging a fluid, such as a pressurized saline solution, directly to a tissue obstruction. The spinal cord stimulator lead can have the form of a percutaneous lead or a surgical lead. Optionally, the spinal cord stimulator lead can include a radiographic marker on the shaft for observation of the stimulator lead under fluoroscopy.

In another aspect, the present invention is a method of implanting a spinal cord stimulator lead in the epidural space. The method includes the steps of inserting a spinal cord stimulator lead having a shaft, a lumen extending through at least a portion of the shaft for carrying a fluid, and a distensible balloon positioned around a distal end of the shaft and in fluid communication with the lumen; inflating and deflating the balloon to displace a tissue obstruction, wherein the balloon expands radially outwardly from at least a portion of the shaft's distal end; and guiding the stimulator lead into a desired position in the epidural space. Preferably, the spinal cord stimulator lead has at least one electrode, and the method further includes delivery of therapeutic energy to tissue adjacent the electrode. Also preferably, the spinal cord stimulator lead can include a second lumen with an outlet at a distal end thereof for injecting fluid into the epidural space to displace a tissue obstruction. Thus, the method can further include the step of injecting fluid, such as a fluid comprising saline, corticosteroid, and/or hyaluronidase, through the stimulator lead to displace a tissue obstruction in the epidural space. Additionally, the method can include the steps of using fluoroscopy to guide placement of the spinal cord stimulator lead and suturing the spinal cord stimulator lead in the desired position in the epidural space.

In yet another aspect, the present invention is a kit. The kit includes a needle, a sterile drape, a fluid coupling, a spinal cord stimulator lead having at least one electrode and a distensible balloon positioned around a distal end of the stimulator lead, and suturing supplies.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a perspective view of a spinal cord stimulator lead having a fluid delivery lumen therethrough in accordance with an example embodiment of the present invention.

FIG. 2 shows a cross-sectional view of a portion of the spinal cord stimulator lead of FIG. 1.

FIG. 3 shows a perspective view of a spinal cord stimulator lead having a fluid delivery lumen extending therethrough, and a balloon for displacing connective tissue in accordance with another example embodiment of the present invention.

FIG. 4 shows a cross-sectional view of a portion of the spinal cord stimulator lead of FIG. 3.

FIG. 5 shows a perspective view of a spinal cord stimulator lead having a fluid delivery lumen extending therethrough, and a balloon for displacing connective tissue in accordance with yet another example embodiment of the present invention.

FIG. 6 shows a cross-sectional view of an end portion of the spinal cord stimulator lead of FIG. 5.

FIG. 7 shows placement of the spinal cord stimulator lead of FIG. 5 in the epidural space according to an example form of the invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

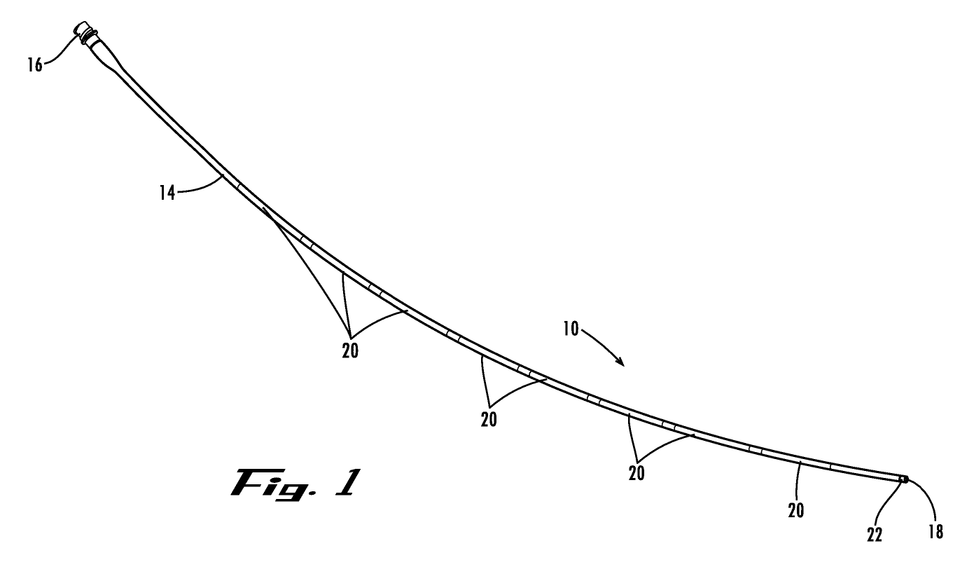

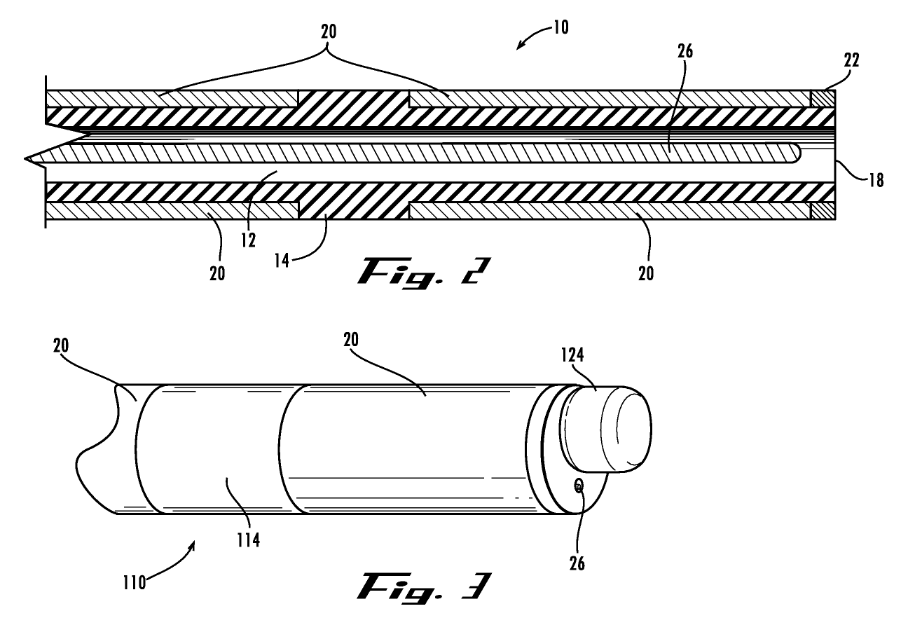

Referring to FIGS. 1 and 2, a spinal cord stimulator lead 10 having a fluid delivery lumen, conduit, or canal 12 extending therethrough is described by way of an example embodiment. The spinal cord stimulator lead 10 can take the form of a percutaneous lead or a surgical paddle lead, for example. Preferably, the spinal cord stimulator lead 10 has a biocompatible, somewhat flexible, electrically non-conductive, cylindrical body or shaft 14. Exemplary materials that can be used to construct the body 14 include, but are not limited to, silicone, polyurethane, or polyethylene. Those skilled in the art will understand that various other biocompatible or biologically inert materials of construction can be used as well, without deviating from the scope of the present invention. The body 14 optionally includes a polyurethane insulation sheath for increased durability and longevity. The fluid delivery lumen or canal 12 preferably extends through or along substantially the entire length of the body 14 for carrying a fluid, such as a saline solution, from a fluid source (not shown) directly to an area of a tissue obstruction or obstructive tissue within the subject's epidural space. As used herein, the terms tissue obstruction and obstructive tissue refer to any fat, vein, adhesion, connective tissue, or other obstruction in the epidural space that interferes with the proper placement of the spinal cord stimulator lead 10. Preferably, the stimulator lead 10 has a connector 16, such as a “leur-lock” type connector, at a proximal end thereof, for connecting to a fluid source to deliver fluid into the lumen 12. Those skilled in the art will understand that various other connectors for connecting the spinal cord stimulator lead 10 to the fluid source can be employed as well without deviating from the scope of the present invention. At the distal tip (i.e., the end opposite the connector 16) is a discharge outlet 18 for discharging fluid from the lumen 12.

The lumen 12 preferably delivers pressurized fluid for direct injection into the area of the tissue obstruction. For example, a saline solution can be injected into the area of the tissue obstruction to help break up the obstruction. In one example embodiment, a mixture of saline, corticosteroid, and hyaluronidase is injected into the site of the tissue obstruction, via the fluid lumen 12 and the outlet 18, to reduce the inflammation. Preferably, the volume of the mixture is not more than about 20 milliliters. Also preferably, the amount of the hyaluronidase is limited to about 150 USP units to no more than about 1500 USP units, while the amount of the corticosteroid administered depends on the type of corticosteroid used. Those skilled in the art will understand how to determine the amount of corticosteroid to administer.

The spinal cord stimulator lead 10 includes at least one, and preferably, a plurality of electrodes 20 for spinal cord stimulation. Preferably, the plurality of electrodes 20 comprises four or eight cylindrical electrodes spaced along the length of the stimulator lead 10. One or more wires or other electrical conductors are preferably embedded in or on the body 14 to deliver electrical signals from an external source to the electrodes 20. FIGS. 1 and 2 show an example embodiment with eight such electrodes 20. In an example embodiment, the stimulator lead includes eight electrodes, each electrode being about 52 mm long, and the stimulator lead being about 60 cm long. In another embodiment, the stimulator lead 10 includes four electrodes 20, each electrode being about 24-34 mm long, and the stimulator lead being about 30 cm long. In example embodiments, the spinal cord stimulator lead 10 has a diameter of about 0.8 mm to about 1.5 mm, though those skilled in the art will understand that the size of the diameter larger or smaller. Those skilled in the art will also understand how to configure the stimulator lead and how to determine, for example, electrode material, size, shape, span, and spacing. Appropriate selection of the stimulator lead size and electrode configuration can be made in accordance with accepted medical protocol as determined by the treating physician.

Optionally, the spinal cord stimulator lead 10 includes a marker 22, such as a radiographic strip or band near the tip of the stimulator lead. The marker can aid the practitioner in guiding the stimulator lead 10 under fluoroscopy or other conventional imaging techniques into a proper placement in the epidural space.

Optionally, the stimulator lead 10 can include a stylet 26 positionable within the fluid lumen 12. Preferably, the stylet 26 is a slender and substantially rigid, but malleable, surgical wire for guiding the stimulator lead 10 into and through the soft tissue. Such use of surgical wire allows the practitioner to view the location of the stylet with conventional imaging technology. The stylet 26 can be straight or can be angled, such as curved at an angle of about 30° to about 45°, for example, to improve steerablilty and control. In instances where the stylet 26 is angled, preferably, the shape of the stimulator lead 10 conforms to angle of the stylet. Preferably, the stylet 26 is removable from the spinal cord stimulator lead 10 such that once the stimulator lead encounters an obstruction, the stylet can be removed and the lumen 12 can be fitted with a connector, such as a male leur-lock connector 16, and coupled to a fluid source for delivering fluid directly to the area of the obstruction. Alternatively, the stylet 26 can extend through a second lumen of the stimulator lead 10 such that the first lumen 12 can be used for fluid injection while simultaneously guiding the stimulator lead with the stylet. Also optionally, a fiber optic scope could be inserted through the lumen for visualization of internal tissue.

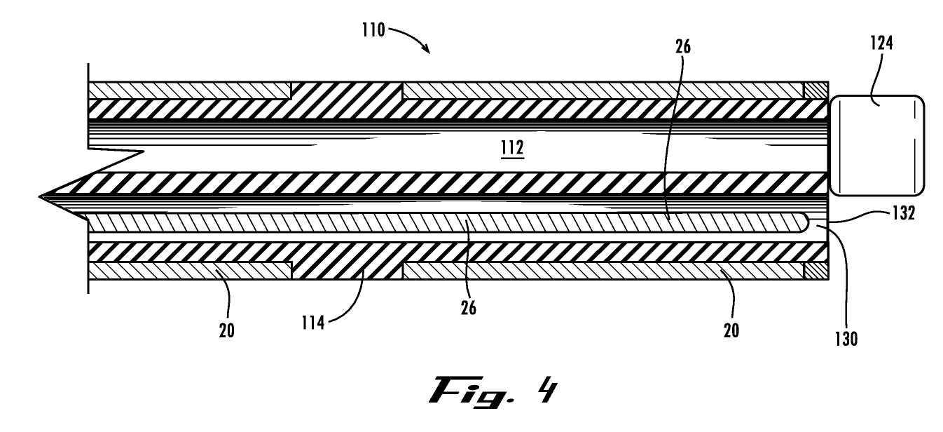

Another example embodiment of the present invention is shown in FIGS. 3 and 4. The spinal cord stimulator lead 110 preferably comprises a plurality of electrodes 20, in substantially similar fashion to the stimulator lead 10 described above with one or more wires or other electrical conductors embedded in or on the body 114 to deliver electrical signals from an external source to the electrode. The spinal cord stimulator lead 110 is substantially similar to the spinal cord stimulator lead 10, but with the exceptions noted herein. The stimulator lead 110 further comprises an inflatable and deflatable balloon 124. The balloon 124 is preferably connected at or near the distal end of the spinal cord stimulator lead 10, in fluid communication with a fluid lumen 112 extending therethrough. The lumen 112 delivers fluid, such as a sterilized liquid or air, from a remote fluid source under sufficient pressure to inflate and deflate the balloon 124. Preferably, the balloon 124 is constructed of a durable, yet distensible, material such as latex, although the present invention also contemplates the use of other distensible, biocompatible materials. The practitioner can alternately inflate and deflate the balloon 124 to displace tissues that prevent the passage or placement of the spinal cord stimulator lead 110. Optionally, the balloon 124 is detachable and retractable through the lumen, so that once the spinal cord stimulator lead 110 is properly placed, the practitioner can disengage the balloon 124 from the stimulator lead and remove it, with, for example, the stylet 26 or some other device. Optionally, the stimulator lead 110 includes a second fluid delivery lumen 130 extending therethrough. In this embodiment, the second lumen 130 is used to deliver fluid directly to the area of the tissue obstruction via discharge outlet 132, while the first lumen 112 is used to deliver fluid to distend the balloon 124. Preferably, the stylet 26 is positionable with the lumen 130 to guide and steer the stimulator lead 110 through the soft tissue and into the epidural space. Alternatively, the stylet 26 can be positionable with a third lumen to guide and steer the stimulator lead 110 through the soft tissue and into the epidural space.

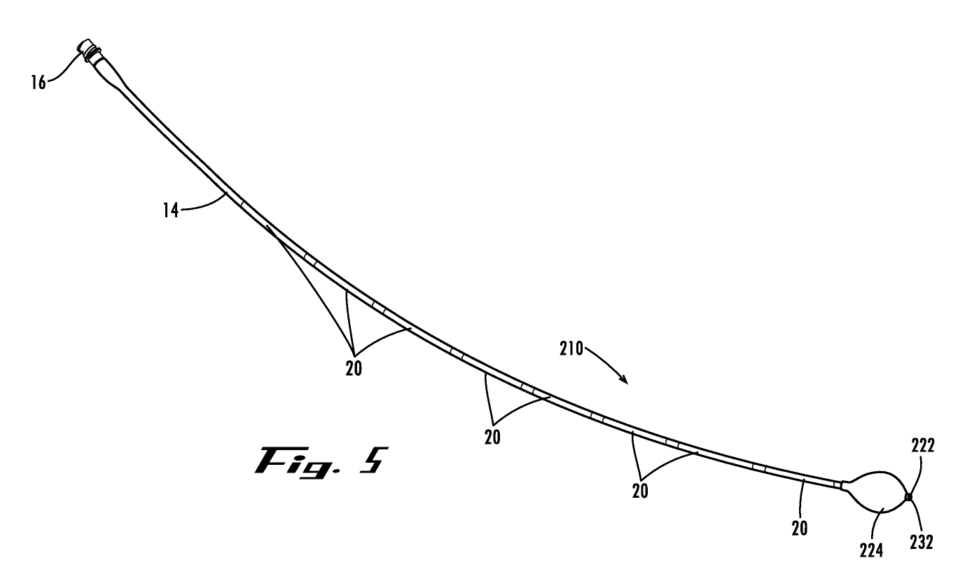

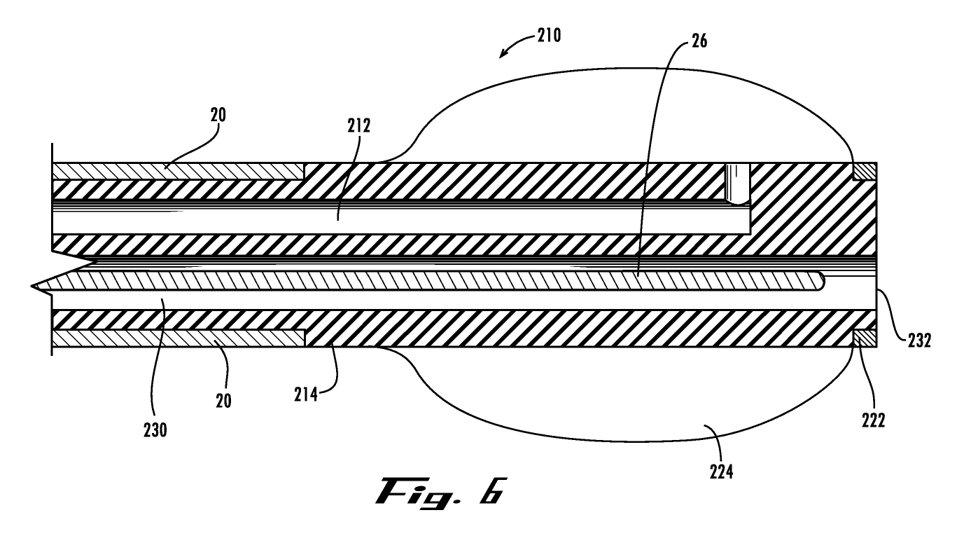

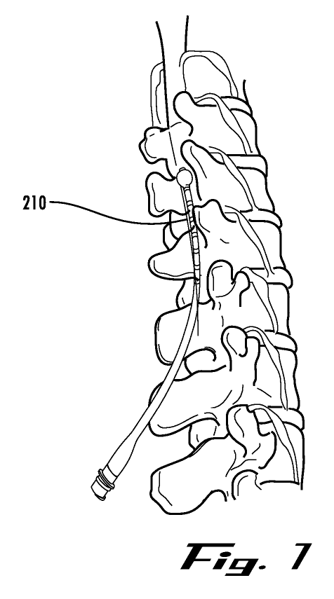

Another example embodiment of the spinal cord stimulator lead 210 is shown in FIGS. 5 and 6. The spinal cord stimulator lead 210 preferably comprises a plurality of electrodes 20, in substantially similar fashion to the stimulator lead 110 described above with one or more wires or other electrical conductors embedded in or on the body 214 to deliver electrical signals from an external source to the electrode. The spinal cord stimulator lead 210 is substantially similar to the spinal cord stimulator lead 110, but with the exceptions noted herein. The spinal cord stimulator lead 210 includes a cuffed balloon 224 located around a distal end thereof. Preferably, the cuffed balloon 224 has a generally oblong shape in the sense that the balloon is longer than it is wide when the balloon is inflated. In an example embodiment, the balloon 224 extends from or near the tip of the shaft or body 214. The cuffed balloon 224 is in fluid communication with the lumen 212 such that the balloon can expand generally radially outwardly about all or a portion of the circumference of the shaft or body 214 at the distal end of the spinal cord stimulator lead 110. Preferably, the balloon 224 can be expanded to a size of about four to six times greater than the diameter of the body 214 of the stimulator lead 110. Thus, preferably, the length of the balloon 224 is at least, and more preferably, greater than four to six times greater than the diameter of the body 214. Preferably, the stimulator lead 210 includes a second lumen 230 or fluid conduit extending therethrough for discharging fluid directing into an area of a tissue obstruction via discharge outlet 232.

The fluid lumen 212 carries a fluid, such as a sterilized liquid or air, under sufficient pressure to inflate and deflate the balloon 224. The diameter of the lumen 212 for delivering a fluid to inflate the balloon 224 is preferably smaller than the diameter of the lumen 230 for carrying a fluid directly to the site of the obstruction. However, those skilled in the art will understand that the lumens 212 and 230 can have substantially the same diameter, or the diameter of the lumen 230 for delivering fluid directly to the site of the obstruction can be smaller than the lumen 212 for carrying fluid to the balloon 224.

Those skilled in the art will also understand that one or both of the fluid lumens 212 and 230 can extend along the outer body of the shaft 214 or within the shaft 214, and the lumen 230 can also serve as the lumen for the stylet 26. Alternatively, a third lumen can serve as the lumen for the stylet 26.

Preferably, the balloon 224 is constructed of a durable, yet distensible, material such as latex, although the present invention also contemplates the use of other distensible, biocompatible materials. The practitioner can alternately inflate and deflate the balloon 224 to laterally displace tissues that prevent the passage or placement of the spinal cord stimulator lead.

In a preferred manner of use, a guide needle is positioned generally in the epidural space of a human or animal subject. The spinal cord stimulator lead 10, 110, or 210 along with the stylet 26 are inserted through the guide needle into the epidural space. Preferably, the practitioner uses fluoroscopy to guide the placement of the guide needle and/or the stimulator lead 10, 110, or 210. As the practitioner is guiding the stimulator lead 10, 110, or 210 into the desired location, the practitioner can remove the stylet 26 and connect the stimulator lead to a fluid source and inject fluid from the fluid source through the lumen 12, 130, or 230 into the epidural space to displace tissue obstructions, which would otherwise interfere with the accurate placement of the electrodes. Optionally, if an embodiment including a distensible balloon 124 or 224 is utilized, the practitioner can direct fluid delivery to expand and contract the balloon for displacement of obstructions. Once the stimulator lead 10, 110, or 210 is positioned as desired in the epidural space of the patient, for example as seen in FIG. 7, the stimulator lead can be secured in place with sutures. The stimulator lead 10, 110, or 210 is disconnected from the fluid source and is connected to a power source for delivery of electrical energy to the electrode(s) 20. The power source may be external, or may be implanted internally, for example in the patient's abdomen or elsewhere. An internal or external controller is preferably used to control the internal power source and activate the electrodes 20 according to a physician prescribed treatment regimen. The spinal cord stimulator lead 10, 110 and 210 thus functions both as a typical catheter when implanting the stimulator lead and as a spinal cord stimulator lead once implanted.

Optionally, the tools and supplies that the practitioner uses to implant the stimulator lead of the present invention into the patient are assembled into a self-contained kit. For example, the kit includes a guide needle, a spinal cord stimulator lead 10, 110, or 210, a sterile drape, a power source, a fluid coupling, and suturing supplies, or any subcombination thereof, within a case or other container.

While the invention has been described with reference to preferred and example embodiments, it will be understood by those skilled in the art that a variety of modifications, additions and deletions are within the scope of the invention, as defined by the following claims.

What is claimed is:

1. A spinal cord stimulator lead for placement in the epidural space of a human or animal subject, the stimulator lead comprising:
a biocompatible shaft, wherein at least a portion of the shaft is flexible;
at least one electrode positioned along the shaft;
a lumen extending through at least a portion of the shaft for carrying a fluid; and
a distensible balloon positioned around a distal end of the shaft and in fluid communication with the lumen.

2. The spinal cord stimulator lead of claim 1, further comprising a second lumen extending through at least a portion of the shaft for discharging a fluid directly to a tissue obstruction.

3. The spinal cord stimulator lead of claim 2, further comprising a leur-lock connector at a proximal end of the shaft for connecting a fluid source into communication with second the lumen.

4. The spinal cord stimulator lead of claim 3, wherein the fluid source delivers a pressurized saline solution.

5. The spinal cord stimulator lead of claim 1, wherein the lumen carries a sterilized fluid under sufficient pressure to expand the balloon.

6. The spinal cord stimulator lead of claim 1, wherein the balloon is formed of latex.

7. The spinal cord stimulator lead of claim 1, further comprising a radiographic marker on the shaft, for observation of the stimulator lead under fluoroscopy.

8. The spinal cord stimulator lead of claim 1, further comprising a stylet for guiding the stimulator lead into and through the epidural space.

9. The spinal cord stimulator lead of claim 8, wherein the stylet is positioned within a second lumen extending through at least a portion of the shaft.

10. The spinal cord stimulator lead of claim 1, wherein the balloon is a cuffed balloon that expands radially outwardly from at least a portion of the shaft's distal end.

11. The spinal cord stimulator lead of claim 1, wherein the spinal cord stimulator lead has the form of a percutaneous lead.

12. The spinal cord stimulator lead of claim 1, wherein the spinal cord stimulator lead has the form of a surgical lead.

13. A method of implanting a spinal cord stimulator lead in the epidural space, comprising:
inserting a spinal cord stimulator lead having a shaft, a lumen extending through at least a portion of the shaft for carrying a fluid, and a distensible balloon positioned around a distal end of the shaft and in fluid communication with the lumen;
inflating and deflating the balloon to displace a tissue obstruction, wherein the balloon expands radially outwardly from at least a portion of the shaft's distal end; and
guiding the stimulator lead into a desired position in the epidural space.

14. The method of claim 13, wherein the spinal cord stimulator lead comprises at least one electrode, said method further comprises delivery of therapeutic energy to tissue adjacent the electrode.

15. The method of claim 13, further comprising the step of using fluoroscopy to guide placement of the spinal cord stimulator lead.

16. The method of claim 13, further comprising suturing the spinal cord stimulator lead in the desired position in the epidural space.

17. The method of claim 13, wherein the spinal cord stimulator lead comprises a second lumen with an outlet at a distal end thereof for injecting fluid into the epidural space to displace a tissue obstruction, said method further comprises injecting fluid through the stimulator lead to displace a tissue obstruction in the epidural space.

18. The method of claim 17, wherein the step of injecting fluid comprises discharging through the outlet a fluid comprising saline, corticosteroid, and/or hyaluronidase into the area of the tissue obstruction.

19. A kit, comprising:
a needle;
a sterile drape;
a fluid coupling;
a spinal cord stimulator lead having at least one electrode and a distensible balloon positioned around a distal end of the stimulator lead; and
suturing supplies.
